(19) United States
(12) Patent Application Publication
(10) Pub. No.: US 2016/0206477 A1
(43) Pub. Date: Jul. 21, 2016

(54) HEARING PROTECTION DEVICE

(71) Applicant: RACAL ACOUSTICS LTD, Harrow, Middlesex (GB)

(72) Inventors: Norman Davidson, Harrow (GB); Habib Hussein, Harrow (GB)

(21) Appl. No.: 14/912,597

(22) PCT Filed: Aug. 14, 2014
(86) PCT No.: PCT/GB2014/052495

§ 371 Date: Feb. 17, 2016

(30) Foreign Application Priority Data
Aug. 19, 2013 (GB) 1314808.5

(51) Int. Cl. A61F 11/08; H04R 1/10

(52) U.S. Cl. CPC A61F 11/08; H04R 1/1083; A61F 2011/085; H04R 2460/01

(57) ABSTRACT

An earpiece for a hearing protection device comprising an earbud (301), which is arranged such that it may be inserted into the ear canal of a user to block the ear canal of the user, wherein an acoustic pathway (303) is provided, which extends through the earpiece, and wherein an acoustic resistor (304) is provided that blocks the acoustic pathway, which acoustic resistor comprises an air permeable material.

The present disclosure relates to a hearing protection device, which is suitable for providing protection against hearing damage in environments with medium or high ambient noise, and to an earpiece therefor.

When an earpiece, which is designed to protect against hearing damage in environments with medium or high ambient noise level, is inserted into a user's ear canal, the objective is typically to totally block the ear canal from the ambient environment.

Blocking one or both ear canals alters a person's perception of their own voice, which phenomenon is commonly referred to as “occlusion effect”. The occlusion effect is caused by the sound of the person's voice entering the blocked ear canal by means of skin and bone conduction. The sound generated by the person's voice then reverberates within the blocked ear canal, causing a frequency and amplitude shift. Normally, these sounds escape out of an unblocked ear canal and a person is not aware of them.

In a blocked ear canal, the low frequency element of a person's voice can be significantly amplified, by up 20 decibels (dB) or more (Reference: Hearing Loss by Mark Ross, Ph.D. February 2004). The occlusion effect can also occur when the jawbone is moved, for example eating and drinking, and the resultant sound is modified.

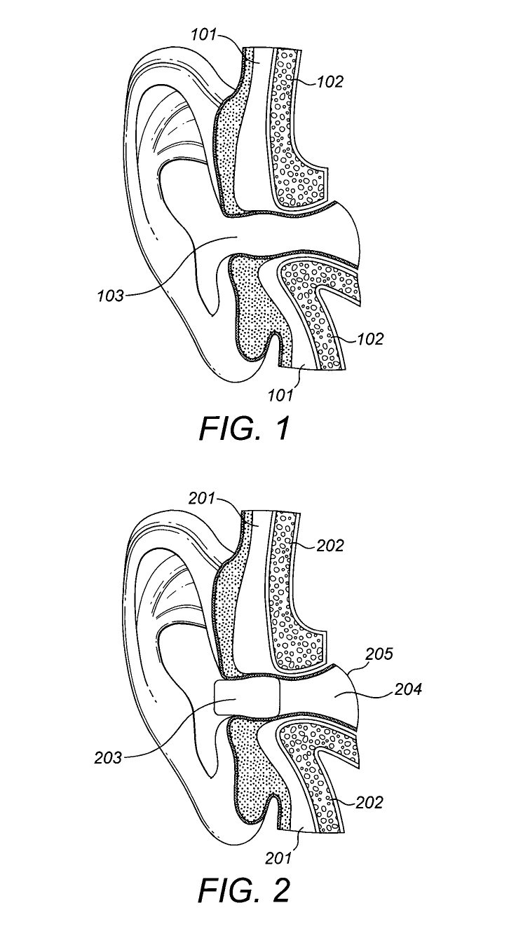

FIG. 1 shows a partial sectional view of an open/unblocked ear canal (103). This represents the normal state of a person's hearing. The individual “hears” their own voice by means of air conduction from the mouth and via skin (101) and bone (102) conduction. A sound entering the ear canal by means of skin (101) and bone (102) conduction escapes out of the un-blocked ear canal (103).

FIG. 2 shows the ear canal depicted in FIG. 1 blocked by a typical prior art high passive attenuation earpiece that consists of an earbud. Hearing protection is achieved by simply blocking the ear canal from the ambient environment with the earbud. A total blocking of the ear canal is preferred for effective hearing protection, since any acoustic path from the ambient environment to the ear drum reduces the hearing protection provided by the device. Typical prior art in-the-ear devices such as that shown may consist of foam, silicon or custom moulded earbuds.

The blocking of the ear canal (204) by the earbud (203) causes the individual's perception of their own voice to change by virtue of the above described occlusion effect. A person's voice enters the blocked ear canal (204) by skin (201) and bone (202) conduction. Their voice then reverberates within the blocked ear canal (204). In the blocked ear canal (204), the skin and bone conduction is a high percentile of the overall sound received at the ear drum (205).

Also, if the earpiece has a lanyard or a cable connected to it, mechanical vibrations can be transmitted into the blocked ear canal (204) and interpreted as noise, hereinafter referred to as “cable noise”.

Furthermore, in the blocked ear canal (204) there is no air path to allow equalisation of air pressure in the blocked ear canal (204) with the ambient environment. The air pressure differential can cause the hearing protection device (203) to be dislodged, which reduces the sound attenuation of the device. Insertion of the earbud (203) can also cause a build-up of air pressure within the blocked ear canal (204) with no means of equalising the air pressure. If the air pressure is not equalised, then over a period of time discomfort can be experienced.

The present invention arose in a bid to provide an improved hearing protection device offering sufficient noise attenuation to provide hearing protection in medium or high noise environments whilst at the same time minimising the occlusion effect and allowing for pressure equalisation.

According to the present invention in a first aspect, there is provided an earpiece for a hearing protection device comprising an earbud, which is arranged such that it may be inserted into the ear canal of a user to block the ear canal of the user, wherein an acoustic pathway is provided, which extends through the earpiece, and wherein an acoustic resistor is provided that blocks the acoustic pathway, which acoustic resistor comprises an air permeable material.

The earpiece protects against hearing damage in medium or high ambient noise environments. The earpiece provides passive attenuation sufficient for such purpose.

The acoustic pathway extends between the ambient environment and the ear canal when the earpiece is in use. The acoustic pathway extends entirely through the earbud. No other acoustic pathway through the earbud exists. The acoustic pathway allows for acoustic leakage. The acoustic leakage is controlled by the acoustic resistor.

The acoustic resistor completely blocks/covers the acoustic pathway. No air may pass through the acoustic pathway (and thereby between the ambient environment and the inner ear canal/eardrum when the earpiece is in use) without travelling through the acoustic resistor.

Further preferred features are presented in the dependent claims.

According to the present invention in a further aspect, there is provided a hearing protection device comprising one or more earpieces as defined above. The hearing protection device preferably comprises a pair of earpieces.

Non-limiting embodiments will now be described, by way of example only, with reference to the accompanying drawings, in which:

FIG. 1 shows a partial sectional view of an open/unblocked ear canal;

FIG. 2 is a view similar to FIG. 1 but showing the ear canal blocked by a prior art earpiece;

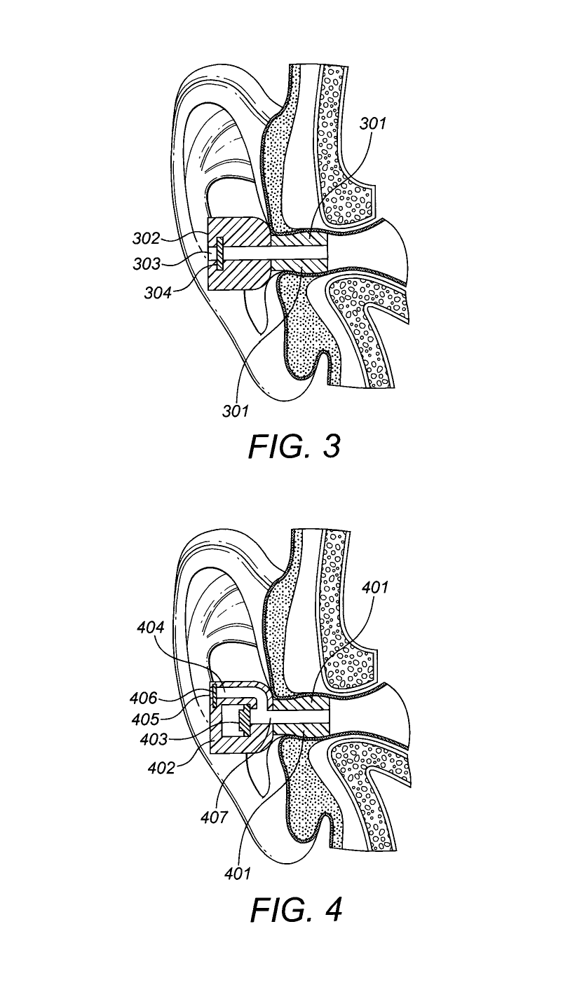

FIG. 3 shows an earpiece according to a first embodiment located within the ear canal;

FIG. 4 shows an earpiece according to a second embodiment located within the ear canal; and

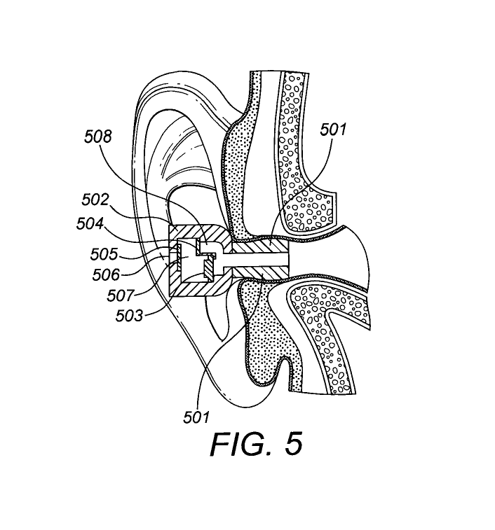

FIG. 5 shows an earpiece according to a third embodiment located within the ear canal.

With reference to FIGS. 3, 4 and 5, there are shown earpieces, for hearing protection devices, in accordance with first to third embodiments of the present invention. In each embodiment, in order to reduce the occlusion effect and to allow for pressure equalisation, an acoustic leakage is introduced into the earpiece. Whilst the introduction of an acoustic leakage would ordinarily cause a significant loss of sound attenuation (making the earpieces useless for protecting against hearing damage in medium or high ambient noise environments), the embodiments of the present invention are uniquely configured to allow for a controlled acoustic leakage, which provides sufficient acoustic leakage for reduction of the occlusion effect and for pressure equalisation whilst at the same time maintaining sufficient attenuation, wherein each embodiment will provide adequate protection against hearing damage for users even in medium or high ambient noise environments.

A medium ambient noise environment is an environment where hearing damage or noise induced hearing loss can occur with long term exposure to the noise. The law of many countries attributes a continuous sound level of 85 dbA to this environment. Hearing damage or noise induced hearing loss can occur after an expose period of 8 hour per day in such an environment. For impulse or impact noise the level is set at 140 db peak sound pressure level (SPL).

A high ambient noise environment is an environment where hearing damage or noise induced hearing loss can occur with short term exposure to the noise. The law of many countries attributes a continuous sound level of 105 dbSPL to this environment. Hearing damage or noise induced hearing loss can occur after an expose period of 1 hour per day.

The above definitions of medium and high ambient noise environments are adopted herein.

Common to each of the embodiments is the provision of an acoustic pathway through the earpiece. The acoustic pathway allows for the flow of air between the ambient environment and the ear drum when the earpiece is in use.

Unique to the present invention is the blocking of the acoustic pathway with an acoustic resistor, which acoustic resistor restores the sound attenuation of the earpiece whilst allowing for airflow therethrough to reduce the occlusion effect and to allow for pressure equalisation. It is the combination of the acoustic pathway and the acoustic resistor blocking the acoustic pathway that provides the controlled acoustic leakage discussed above.

A further benefit of the present invention, when the earpiece comprises a cable attached thereto, is the reduction of cable noise.

Considering the first embodiment as shown in FIG. 3, there is provided an earpiece for a high passive attenuation in-the-ear device. The earpiece comprises an earbud (301), which is sized and shaped to block the ear canal (305) and a housing (302) to which the earbud is sealingly attached.

The earbud (301) may be formed from any material commonly used for the production of typical prior art passive sound attenuating earbuds, such as silicon or foam. The earbud may be formed into a range of standard shapes and sizes or may be custom moulded. The earbud may be unitarily formed from the resilient material or may be provided with a central reinforcing member, which comprises a rigid plastic tube or similar element, around which the resilient material is moulded. The earbud is provided with a through hole, which forms part of an acoustic path (303) through the earpiece.

The housing (302) may be formed from a rigid plastic, such as ABS (Acrylonitrile Butadiene Styrene). Whilst the housing is shown to be substantially solid it may comprise a substantially hollow shell. The housing (302) is provided with a through hole, which forms part of the acoustic path (303) through the earpiece and which is in communication with the through hole in the earbud. The housing and the earbud may be joined to one another by adhesive or using any other suitable method, as will be readily appreciated by those skilled in the art. One particular possibility is for the housing to be formed with a spigot, which comprises a through hole to form part of the acoustic path through the earpiece, the spigot being adapted to be received by the through hole of the earbud for attaching the housing and earbud to one another. The engagement of the spigot by the through hole in the earbud may be provided by an interference or screw fit or otherwise.

The acoustic path (303) extends fully through the earpiece to provide the acoustic pathway between the ambient environment and the blocked ear canal and thereby allowing for the desired acoustic leakage.

In accordance with the arrangement of FIG. 3, the acoustic path comprises a through hole that extends continuously through both the earbud and the housing. The acoustic path may, as shown, comprise a through hole of constant diameter/open area or may alternatively comprise a through hole of varying diameter/open area. It may be round, square or otherwise shaped and need not be of constant shape. In arrangements where the housing comprises a substantially hollow shell, the shell may be provided with a first opening that is in communication with the through hole in the earbud and a second opening that is in communication with the ambient environment, wherein a void in the housing between the first and second openings forms part of the acoustic path. Numerous alternative configurations are available, as will be readily appreciated by those skilled in the art.

In further alternative arrangements, the earpiece may comprise an earbud only, i.e. the housing need not be provided. The through hole forming the acoustic path may be formed during the moulding of the earbud or may be formed in the central reinforcing member if provided. Here the earbud may be extended axially from that shown. As will be appreciated any suitable desired form of earbud may be produced by moulding.

Irrespective of the specific configuration of the earpiece and the acoustic path, the acoustic path provides an acoustic pathway between the ambient environment at a first end, and user's ear canal at a second end, as shown.

Within the acoustic path there is fixed an acoustic resistor. The acoustic resistor comprises air permeable material (304), which is arranged to extend completely across the acoustic path such that no air may pass through the acoustic path between the ambient atmosphere and the inner ear canal without passing through the acoustic resistor. The air permeable material most preferably comprises a porous material, in particular a porous membrane, although may be provided in other forms.

The permeable material may have a larger surface area than the open area of the acoustic path at the point along the acoustic path at which the permeable material is fixed so that the fixing of the permeable material is made simple whilst the acoustic path is completely covered/blocked by the permeable material. In the arrangement of FIG. 3, the permeable material is fixed in place during moulding of the housing (302) with the portion of the permeable material that extends radially beyond the acoustic path retained by the material of the housing that is moulded around it. In the arrangement of FIG. 3, the permeable material comprises a disc having a larger diameter than the acoustic path, which has a circular cross section. Where the housing is substantially hollow, a piece of permeable material with a greater surface area may be fixed over one or both of the first and second openings that form part of the acoustic path. Here the permeable material may be attached using adhesive or double sided tape. A circular recess having a greater diameter than the opening may be provided around either or both of the first and second openings with a disc of permeable material adhered to the recess using the adhesive or double sided tape.

Whilst the acoustic resistor is shown to be of single piece construction, comprising a single piece of permeable material, as mentioned above, it is also possible that two or more separate pieces of the permeable material are provided to form the acoustic resistor, wherein the attenuation properties of the separate pieces of the permeable material are added together. Multiple pieces of the permeable material may be used in any of the arrangements described above, wherein the separate pieces of material may be provided as a laminate or may be spaced from one another along the acoustic path.

In any of the above described arrangements the permeable material may be used on its own or, alternatively, there may be a protective cover applied to the permeable material, such as a nylon mesh, which has substantially no acoustic attenuation capability but which shields the permeable material and prevents damage to the permeable material. The protective material may have the same footprint as the permeable material.

The air permeable material (304) blocking the acoustic path has a high enough acoustic impedance, measured in RAYLs, to maintain the sound attenuation of the earpiece. The RAYL value of the air permeable material comprising the acoustic resistor, as will be fully appreciated by those skilled in the art, may be calculated using the following formulae:

$$Rs = \frac{p}{v}$$

where: Rs is the value in RAYL s of the specific acoustic impedance
p is the sound pressure
v is the particle velocity

Rc=dc

where: Rc is the value in RAYL s of the characteristic acoustic impedance;
d is the density of the material; and
c is the speed of sound

For measurement of the RAYL value of the air permeable material comprising the acoustic resistor, the test method specified in ASTM C384 (2003)—“Standard Test Method for Impedance and Absorption of Acoustical Materials by the Impedance Tube Method” is followed.

RAYLs can be expressed in two different units, MKS and CGS where 1 CGS RAYLs is equal to 10 MKS RAYLs

Particularly preferred porous materials for use in the present invention are porous membranes, in particular Cyclopore™ membranes, commercially available from Whatman Ltd.

Such membranes are available in a range of pore sizes, the pore sizes of the different membranes ranging from 0.1 to 12 μm. Further parameters of the different membranes are as follows:

### Thickness: 7 to 20 μm

Weight: 0.7 to 2.0 mg/cm²
Porosity (void vol.): 4 to 20%
Pore density: 10⁵ to 6×10⁸ pores/cm²
Specific gravity: 1.21 g/cm²

The membranes are obtained in the form of discs having a diameter of 25 or 47 mm. These discs are cut or stamped to provide suitably sized/shaped membrane pieces for use in the production of earpieces according to the present invention.

In a specific example, constructed in accordance with the arrangement of FIG. 3 and in which the acoustic resistor comprises a single piece of the porous membrane, the acoustic path, and thereby the portion of the porous membrane that blocks the acoustic path, has a diameter of 1.5 mm. The portion of porous membrane blocking the acoustic path has a cross sectional area of 1.7 mm². The porous membrane comprises a Cyclopore™ Polycarbonate Membrane (part number 7060-4713) from Whatman Ltd. The acoustic resistor has an acoustic impedance of at least 5,000 RAYLs MKS, preferably at least 8,000 RAYLs MKS. With such a configuration, a minimum sound attenuation of 30 decibels (dB) across the audio spectrum can be achieved by a correctly fitted earpiece.

The passive earpieces described above will find use in a range of fields/situations requiring hearing protection against medium or high noise levels, including, the operation of industrial equipment and sport shooting, etc.

Whilst the above described passive earpieces feature no electronics, it will be desirable in some situations to introduce electronics into the earpieces, which electronics may allow for active noise reduction to supplement the passive noise reduction of the earpieces or to provide communications capabilities, environmental talk-though capabilities or other features. For example, in many military applications hearing protection is required from the noise of gunfire, explosions, armored vehicles, etc, yet there is a need to remain in communication with comrades at all times. The passive earpieces described above can be modified with the introduction of electronic transducers to enable these features. The introduction of such transducers affects the acoustic pathways within the earpieces. Therefore, the controlled acoustic leakage must be adjusted accordingly.

Exemplary embodiments, which introduce transducers are shown in FIGS. 4 and 5.

FIG. 4 shows an earpiece (401, 402) that comprises a sound reproduction transducer (403), which comprises a speaker. Here, the housing is formed by moulding from a rigid plastic and comprises a substantially hollow shell, as described above. The earbud may be formed/attached in any manner as described above in respect of the first embodiment. The attachment is most preferably through the described spigot arrangement.

The housing is provided with a first opening (407) that is in communication with the through hole in the earbud and a second opening (405) that is in communication with the ambient environment, wherein a void or passage (404) in the housing between the first and second openings forms part of the acoustic path through the earpiece.

The sound reproduction transducer (403) is provided with a rear void, which may be considered to provide a transducer area. The transducer area of this embodiment is closed to environment by the speaker (403), which covers its front. The acoustic path bypasses the transducer area (402) of the device. Using this method partially isolates the acoustic path (404) from the introduced transducer and its associated acoustics. This simplifies the design of the acoustic path. The acoustic resistor comprises a single disc like piece of permeable material, which blocks the acoustic path and restores the acoustic attenuation of the earpiece. Any permeable material described in accordance with the first embodiment may be used. The permeable material may be mounted in any manner as described above in respect of the first embodiment. Moreover, as will be readily appreciated in accordance with the discussions above in respect of the first embodiment, alternative acoustic resistor configurations are possible, which feature multiple pieces of the permeable material.

FIG. 5 shows a further alternative earpiece that again comprises a sound reproduction transducer (503), which comprises a speaker. The construction of the earpiece is substantially in accordance with the embodiment of FIG. 4. In contrast to the embodiment of FIG. 4, however, the acoustic path incorporates the transducer and its associated acoustics into its path (508, 507, 506).

The permeable material placed across the acoustic path, which provides the acoustic resistor, is split into two separate pieces. A first material piece (504), which blocks/covers the portion of the acoustic path (508) from the front of the transducer to the rear of the transducer, and a second material piece (505), which blocks/covers the portion of the acoustic path (506, 507) from the rear of the acoustic transducer to the ambient environment. Here, therefore, the controlled acoustic leakage from the blocked ear canal passes through the earpiece by travelling in turn through the through hole in the earbud (501), the acoustic path (508), the first permeable material piece (504), the transducer acoustics (507), the second permeable material piece (505) and the acoustic path (506) to the ambient environment. Any permeable material described in accordance with the first embodiment may be used. The permeable material may be mounted in any manner as described above in respect of the first embodiment. It should be appreciated that in alternative arrangements in accordance with the present embodiment one of the permeable material pieces could be omitted.

A further possible development that may be made to an earpiece in accordance with the second or third embodiments, by virtue of their controlled acoustic leakage, is the introduction of feed forward noise reduction. The acoustic leakage provides a method of stabilizing the frequency response of the speaker and thereby the noise reduction filter profile, whilst the acoustic resistor prevents the passive sound attenuation being diminished.

Conventional noise reduction components/circuitry may be introduced into the earpiece, as will be readily appreciated by those skilled in the art. Such circuitry may comprise a feed forward circuit and a microphone. The microphone is positioned in the housing such that it may detect the noise appearing at the user's ear. The signal detected by the microphone is inverted and added to the drive signal of the speaker, creating the cancellation signal. This active attenuation combined with the passive attenuation, provides a particularly beneficial result.

Numerous alternatives and modifications within the scope of the appended claims will be readily appreciated by those skilled in the art.

1. An earpiece for a hearing protection device comprising an earbud, which is arranged such that it may be inserted into the ear canal of a user to block the ear canal of the user, wherein an acoustic pathway is provided, which extends through the earpiece, and wherein an acoustic resistor is provided that blocks the acoustic pathway, which acoustic resistor comprises an air permeable material.

2. An earpiece as claimed in claim 1, wherein the acoustic resistor has an acoustic impedance of at least 5000 RAYLs MKS.

3. An earpiece as claimed in claim 1, wherein the acoustic resistor has an acoustic impedance of at least 8000 RAYLs MKS.

4. An earpiece as claimed in claim 1, wherein the acoustic resistor comprises a single piece of the air permeable material

5. An earpiece as claimed in claim 1 comprising two or more separate pieces of the air permeable material, which are provided as a laminate or are spaced from one another within the acoustic pathway.

6. An earpiece as claimed in claim 1, wherein the air permeable material comprises a porous material.

7. An earpiece as claimed in claim 6, wherein the air permeable material comprises a porous membrane.

8. An earpiece as claimed in claim 6, wherein the porous material has a pore size of 0.1 to 12 μm.

9. An earpiece as claimed in claim 6, wherein the porous material has a porosity of between 4 and 20%.

10. An earpiece as claimed in claim 6, wherein the porous material has a pore density of between 105 and 6×108 pores per cm2.

11. An earpiece as claimed in claim 1, wherein the air permeable material has a thickness of 7 to 23 μm.

12. An earpiece as claimed in claim 1, wherein the air permeable material comprises a Cyclopore™ membrane.

13. An earpiece as claimed in claim 1, wherein the earpiece further comprises a sound reproduction transducer.

14. An earpiece as claimed in claim 13, wherein the earpiece further comprises a feed forward circuit and a microphone, which are configured to provide a real time anti-phase acoustic signal through the sound reproduction transducer to thereby provide active noise reduction.

15. An earpiece as claimed in claim 14, wherein the sound reproduction transducer is arranged such that the acoustic leakage through the acoustic pathway stabilizes the frequency response of the sound reproduction transducer.

16. An earpiece as claimed in claim 1, wherein the acoustic pathway is provided by an acoustic path that extends through the earbud, wherein, in use, a first end of the acoustic path at a first end of the earbud is open to ambient atmosphere and a second end of the acoustic path at the opposite end of the earbud opens into the user's ear canal.

17. An earpiece as claimed in claim 1 further comprising a housing that is sealingly attached to the earbud, the acoustic pathway being provided by an acoustic path that extends through the earbud and the housing, wherein, in use, the end of the acoustic path at the housing is open to ambient atmosphere and the end of the acoustic path at the earbud opens into the user's ear canal.

18. (canceled)

19. (canceled)
